SEQUENCE LISTING (40 sequences)

SEQ ID NO: 1 - DNA (genomic), 1716 bp; single-stranded, linear
  CDS 153..1625: /codon_start= 153
aaaagcattatgttttgaggacacaaccaacccccaccccacccctcatttctttgacctgcatattaatattgttgtgaatcttggcattacttattttttattatgggagcgttcttcatatttgaatttttatttttgaagcaataaatatgttaattatagtcttattatttttagctactttagctaattccctcgattgtagcggagatgtattttttggatatactagaggagacaaaacagatgttcataaaagtcaagcattaacagcggttaaaaatattaaaagatggcttggaagttttgaaacaagacagtcctttaaagtaatagaaggtgatattgctggttttgcttgggtaggtagttatattaagaactctgattttgttgataatgtaattgagattatgtataatgaagtaaataaaaatggtataccagttgaactttatattgagaatatcgttgataacgagccaggtaaatcttttggttttattcttaattctcataaaaacttagaaaatgctcaaaaagcagttaaaaactggagtactggggttaagtataatgtttatgaaggtaataagatttataaagatcattccgtttgttatttagatgagtctaagaaaaagccagaagctaacgataaggaagcaggtgaatgttattatacaagactaggagataattctaatccctatactcaagttaaaactcctaagccatatttagatgtattcaattctaataatttaactaaaatagttagtggtgaagctttttgttattctgagggcagtttacctgatgtgggtatatgtgttcctattaagtctaatatggattttaaatattataataaatcacctaaacaagatctggataaacagaaagtaattaacgctttaaatactttaagtaaaaattttactgaatctgaaaatcgtcaatcttttatttatcaaaaagataatatagtgggctatatgtggttaggtcaaagaataaataatactgaaaatttgtttaattcattaacaaatgaggtaactaaaaatggagttccagaccatttttattatgaatatgctaaaaatgatcctatgatacaaataggtatttttattaataaacaaggtaatgtagatttagctaaacaggtaggtaaagtttggtctactggtaaacaatttaataatattactggtaaaaagtcgattagtactagtttttgtatattagataataaagaaaaaagaggatttactaatgattatagtgttggacaatgtttaaactttacttatgaagaaaatgtaaatgtaggattaactgatgaaattcttgttgaatataatcctggtttttatagtgctaattatggtgacactttatgtaagagtattggttatcctccttctaataaacctataaaggattattgtaagttttatattgtacaagaagatgatacttgtgttagtatagcttctaaatatccaggattaaccgaacaagatataattgattataattcaaagaacggtgacttttatggatgttttaatctatgggaaggtgataagatttgtatatctaaaccttacatgtaatacttttgattttactgtcagagttactattgtcattaatagtattaaacttcttattttcagtgaattctattaattttgcgtgatc

SEQ ID NO: 2 - protein, 491 aa
MLIIVLLFLATLANSLDCSGDVFFGYTRGDKTDVHKSQALTAVKNIKRWLGSFETRQSFKVIEGDIAGFAWVGSYIKNSDFVDNVIEIMYNEVNKNGIPVELYIENIVDNEPGKSFGFILNSHKNLENAQKAVKNWSTGVKYNVYEGNKIYKDHSVCYLDESKKKPEANDKEAGECYYTRLGDNSNPYTQVKTPKPYLDVFNSNNLTKIVSGEAFCYSEGSLPDVGICVPIKSNMDFKYYNKSPKQDLDKQKVINALNTLSKNFTESENRQSFIYQKDNIVGYMWLGQRINNTENLFNSLTNEVTKNGVPDHFYYEYAKNDPMIQIGIFINKQGNVDLAKQVGKVWSTGKQFNNITGKKSISTSFCILDNKEKRGFTNDYSVGQCLNFTYEENVNVGLTDEILVEYNPGFYSANYGDTLCKSIGYPPSNKPIKDYCKFYIVQEDDTCVSIASKYPGLTEQDIIDYNSKNGDFYGCFNLWEGDKICISKPYM

SEQ ID NO: 3 - DNA (other nucleic acid), 65 bp; single-stranded, linear
  description: /desc = “Synthetic”
aattcatgttaattatagtcttattatttttagctactttagctaattccctcgattgtagcgga

SEQ ID NO: 4 - DNA (other nucleic acid), 48 bp; single-stranded, linear
  description: /desc = “Synthetic”
gatgtattttttggatatactagaggagacaaaacagatgttcataaa

SEQ ID NO: 5 - DNA (other nucleic acid), 35 bp; single-stranded, linear
  description: /desc = “Synthetic”
agtcaagcattaacagcggttaaaaatattaaacg

SEQ ID NO: 6 - DNA (other nucleic acid), 37 bp; single-stranded, linear
  misc_feature 1: /product= “5′ Phosphate”
  description: /desc = “Synthetic”
taaagtagctaaaaataataagactataattaacatg

SEQ ID NO: 7 - DNA (other nucleic acid), 48 bp; single-stranded, linear
  misc_feature 1: /product= “5′ phosphate”
  description: /desc = “Synthetic”
tctagtatatccaaaaaatacatctccgctacaatcgagggaattagc

SEQ ID NO: 8 - DNA (other nucleic acid), 63 bp; single-stranded, linear
  misc_feature 1: /product= “5′ phosphate”
  description: /desc = “Synthetic”
catgcgtttaatatttttaaccgctgttaatgcttgacttttatgaacatctgttttgtctcc

SEQ ID NO: 9 - DNA (other nucleic acid), 44 bp; single-stranded, linear
  description: /desc = “Synthetic”
cgcgaattcgacagaatgttaattatagtcttattatttttagc

SEQ ID NO: 10 - DNA (other nucleic acid), 42 bp; single-stranded, linear
  description: /desc = “Synthetic”
cgtctccggtcctcttttaatatttttaaccgctgttaatgc

SEQ ID NO: 11 - DNA (other nucleic acid), 31 bp; single-stranded, linear
  description: /desc = “Synthetic”
cgcgaattcggttccttatcaagctgacttg

SEQ ID NO: 12 - DNA (other nucleic acid), 39 bp; single-stranded, linear
  description: /desc = “Synthetic”
gttaaaaatattaaaagaggaccggagacgctctgcggg

SEQ ID NO: 13 - DNA (other nucleic acid), 390 bp; single-stranded, linear
  description: /desc = “Synthetic”
cgcgaattcgacagaatgttaattatagtcttattatttttagctactttagctaattccctcgattgtagcggagatgtattttttggatatactagaggagacaaaacagatgttcataaaagtcaagcattaacagcggttaaaaatattaaaagaggaccggagacgctctgcggggctgagctcgtggatgctctgcagttcgtgtgtggagacaggggcttttatttcaacaagcccacagggtatggctccagcagtcgacgggcgcctcagacaggcatcgtggatgagtgctgcttccggagctgtgatctaaggaggctcgagatgtattgcgcacccctcaagcctgccaagtcagcttgataaggaaccgaattccgc

SEQ ID NO: 14 - protein, 118 aa
MLIIVLLFLATLANSLDCSGDVFFGYTRGDKTDVHKSQALTAVKNIKRGPETLCGAELVDALQFVCGDRGFYFNKPTGYGSSSRRAPQTGIVDECCFRSCDLRRLEMYCAPLKPAKSA

SEQ ID NO: 15 - protein, 5 aa
GTEFR

SEQ ID NO: 16 - DNA (genomic), 341 bp; single-stranded, linear
gaattccccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttccttctcgtacgagcttgctcctgatcagcctatctcgcagctgatgaatatctgtggtaggggtttgggaaaatcattcgagtttgatgtttttcttggtatttcccactcctcttcagagtacagaagattaagtgagacgttcgtttgtgcaagctt

SEQ ID NO: 17 - DNA (genomic), 163 bp; single-stranded, linear
tcgagataagctgggggacattcgcgaaaatgaaacaagtcggctgttatagtatatttattataatattgaaagatctcaaaagactacttatttttgaatgaaccaagtatgaaatcaacctatttggggttgaccaaaataagtaaatattaattgtcga

SEQ ID NO: 18 - DNA (genomic), 1018 bp; single-stranded, linear
aagctgactcatgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacaccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagttttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgaggaattc

SEQ ID NO: 19 - DNA (genomic), 2659 bp; single-stranded, linear
ggatctcctgatgactgactcactgataataaaaatacggcttcagaatttctcaagactacactcactgtccgacttcaagtatgacatttcccttgctacctgcatacgcaagtgttgcagagtttgataattccttgagtttggtaggaaaagccgtgtttccctatgctgctgaccagctgcacaacctgatcaagttcactcaatcgactgagcttcaagttaatgtgcaagttgagtcatccgttacagaggaccaatttgaggagctgatcgacaacttgctcaagttgtacaataatggtatcaatgaagtgattttggacctagatttggcagaaagagttgtccaaaggatgatcccaggcgctagggttatctataggaccctggttgataaagttgcatccttgcccgctaatgctagtatcgctgtgcctttttcttctccactgggcgatttgaaaagtttcactaatggcggtagtagaactgtttatgctttttctgagaccgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggttgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaattatttagagattttaacttacatttagattcgatagatcc

SEQ ID NO: 20 - protein, 844 aa
MTFPLLPAYASVAEFDNSLSLVGKAVFPYAADQLHNLIKFTQSTELQVNVQVESSVTEDQFEELIDNLLKLYNNGINEVILDLDLAERVVQRMIPGARVIYRTLVDKVASLPANASIAVPFSSPLGDLKSFTNGGSRTVYAFSETAKLVDVTSTVASGIIPIIDARQLTTEYELSEDVKKFPVSEILLASLTTDRPDGLFTTLVADSSNYSLGLVYSSKKSIPEAIRTQTGVYQSRRHGLWYKGATSGATQKLLGIELDCDGDCLKFVVEQTGVGFCHLERTSCFGQSKGLRAMEATLWDRKSNAPEGSYTKRLFDDEVLLNAKIREEADELAEAKSKEDIAWECADLFYFALVRCAKYGVTLDEVERNLDMKSLKVTRRKGDAKPGYTKEQPKEESKPKEVPSEGRIELCKIDVSKASSQEIEDALRRPIQKTEQIMELVKPIVDNVRQNGDKALLELTAKFDGVALKTPVLEAPFPEELMQLPDNVKRAIDLSIDNVRKFHEAQLTETLQVETCPGVVCSRFARPIEKVGLYIPGGTAILPSTSLMLGVPAKVAGCKEIVFASPPKKDGTLTPEVIYVAHKVGAKCIVLAGGAQAVAAMAYGTETVPKCDKIFGPGNQFVTAAKMMVQNDTSALCSIDMPAGPSEVLVIADKYADPDFVASDLLSQAEHGIDSQVILLAVDMTDKELARIEDAVHNQAVQLPRVEIVRKCIAHSTTLSVATYEQALEMSNQYAPEHLILQIENASSYVDQVQHAGSVFVGAYSPESCGDYSSGTNHTLPTYGYARQYSGVNTATFQKFITSQDVTPEGLKHIGQAVMDLAAVEGLDAHRNAVKVRMEKLGLI

SEQ ID NO: 21 - protein, 48 aa
MLIIVLLFLATLANSLDCSGDVFFGYTRGDKTDVHKSQNLTAVKNIKR

SEQ ID NO: 22 - protein, 48 aa
MLIIVLLFLATLANSLDCSGDVFFGYTRGDKTDVHKSQNLTAVPMYKR

SEQ ID NO: 23 - protein, 48 aa
MLIIVLLFLATLANSLDCSGDVFFGYTRGDKTDVHKSQALTAVPMYKR

SEQ ID NO: 24 - DNA (other nucleic acid), 68 bp; single-stranded, linear
  description: /desc = “Synthetic”
aattcgacagaatgttaattatagtcttattatttttagctactttagctaattccctcgattgtagc

SEQ ID NO: 25 - DNA (other nucleic acid), 54 bp; single-stranded, linear
  description: /desc = “Synthetic”
ggagatgtattttttggatatactagaggagacaaaacagatgttcataaaagt

SEQ ID NO: 26 - DNA (other nucleic acid), 48 bp; single-stranded, linear
  description: /desc = “Synthetic”
ggtccagaaaccttgtgtggtgctgaattggtcgatgctttgcaattc

SEQ ID NO: 27 - DNA (other nucleic acid), 55 bp; single-stranded, linear
  description: /desc = “Synthetic”
gtttgtggtgacagaggtttctacttcaacaagccaaccggttacggttcttctt

SEQ ID NO: 28 - DNA (other nucleic acid), 45 bp; single-stranded, linear
  description: /desc = “Synthetic”
ctagaagagctccacaaaccggtatcgttgacgaatgttgtttca

SEQ ID NO: 29 - DNA (other nucleic acid), 50 bp; single-stranded, linear
  description: /desc = “Synthetic”
aattagctaaagtacgtaaaaataataagactataattaacattctgtcg

SEQ ID NO: 30 - DNA (other nucleic acid), 56 bp; single-stranded, linear
  description: /desc = “Synthetic”
atctgttttgtctcctctagtatatccaaaaaatacatctccgctacattcgaggg

SEQ ID NO: 31 - DNA (other nucleic acid), 48 bp; single-stranded, linear
  description: /desc = “Synthetic”
gtcaccacaaacgaattgcaaagcatcgaccaattcagcaccacacaa

SEQ ID NO: 32 - DNA (other nucleic acid), 57 bp; single-stranded, linear
  description: /desc = “Synthetic”
ggtttctggacctcttttaatatttttaaccgctgttaagttttgacttttatgaac

SEQ ID NO: 33 - DNA (other nucleic acid), 33 bp; single-stranded, linear
  description: /desc = “Synthetic”
caagcattaacagcggttccaatgtacaaaaga

SEQ ID NO: 34 - DNA (other nucleic acid), 57 bp; single-stranded, linear
  description: /desc = “Synthetic”
ggtttctggacctcttttgtacattggaaccgctgttaatgcttgacttttatgaac

SEQ ID NO: 35 - DNA (other nucleic acid), 33 bp; single-stranded, linear
  description: /desc = “Synthetic”
caaaacttaacagcggttccaatgtacaaaaga

SEQ ID NO: 36 - DNA (other nucleic acid), 55 bp; single-stranded, linear
  description: /desc = “Synthetic”
gagctcttctagaagaagaaccgtaaccggttggcttgttgaagtagaaacctct

SEQ ID NO: 37 - DNA (other nucleic acid), 37 bp; single-stranded, linear
  description: /desc = “Synthetic”
gatctgaaacaacattcgtcaacgataccggtttgtg

SEQ ID NO: 38 - DNA (other nucleic acid), 69 bp; single-stranded, linear
  description: /desc = “Synthetic”
aattcttatcaagcagacttagctggcttcaatggagcacagtacatttccaatcttctcaagtcacag

SEQ ID NO: 39 - DNA (other nucleic acid), 33 bp; single-stranded, linear
  description: /desc = “Synthetic”
caaaacttaacagcggttaaaaatattaaaaga

SEQ ID NO: 40 - DNA (other nucleic acid), 57 bp; single-stranded, linear
  description: /desc = “Synthetic”
ggtttctggacctcttttgtacattggaaccgctgttaagttttgacttttatgaac

(19) United States
(12) Patent Application Publication
(10) Pub. No.: US 2003/0013154 A1
(43) Pub. Date: Jan. 16, 2003

(54) Pichia secretory leader for protein expression

(75) Inventors: Kenneth Crawford, Alameda, CA (US); Isabel Zaror, Orinda, CA (US); Robert J. Bishop, San Francisco, CA (US); Michael A. Innis, Moraga, CA (US)

Correspondence Address: Chiron Corporation, Intellectual Property, P.O. Box 8097, Emeryville, CA, 94662-8097, US

(73) Assignee: Chiron Corporation, Emeryville, CA

(21) Appl. No.: 10/179,046

(22) Filed: Jun. 25, 2002

Related U.S. Application Data
(63) Continuation of application No. 09/609,438, filed on Jul. 3, 2000, now abandoned.
Application No. 09/609,438 is a continuation of application No. 09/029,267, filed on Feb. 24, 1998, now Pat. No. 6,107,057.

(51) Int. Cl. C12P 21/02; C12N 9/50; C12N 1/18; C07H 21/04; C12N 15/74

(52) U.S. Cl. 435/69.1; 435/254.2; 435/219; 435/320.1; 536/23.2

(57) ABSTRACT

Polynucleotides, vectors and host cells comprising a polynucleotide having a fragment of a leader sequence and a second nucleotide sequence that encodes a polypeptide heterologous to the leader sequence, wherein the leader sequence fragment is sufficient for secretion and comprises an amino acid sequence that comprises at least about 70% sequence identity to the leader sequence of Pichia acaciae killer toxin, wherein the heterologous polypeptide is not naturally contiguous to the leader sequence, and wherein upon expression of the polynucleotide molecule in a host cell suitable for expression thereof, the heterologous polypeptide is produced that is free of additional N-terminal amino acids.

### CROSS-REFERENCE TO RELATED APPLICATION

This application is a continuation application of copending U.S. application Ser. No. 09/029,267, filed Feb. 24, 1998, the contents of which are herein incorporated by reference in their entirety.

### FIELD OF THE INVENTION

The present invention relates to the production of recombinant polypeptides in host cells, more particularly to compositions and methods for expression and secretion of heterologous proteins.

### BACKGROUND OF THE INVENTION

Recombinant DNA technology has revolutionized the ability to produce polypeptides economically. Yeast host cells and expression systems are useful for such production. Examples of yeast expression systems are Brake, U.S. Pat. No. 4,870,008; Cregg, U.S. Pat. No. 4,837,148; Stroman et al., U.S. Pat. No. 4,855,231; Stroman et al., U.S. Pat. No. 4,879,231; Brierley et al., U.S. Pat. No. 5,324,639; Prevatt et al., U.S. Pat. No. 5,330,901; Tschopp, EP 256 421; Sreekrishna et al., J. Basic Microbiol. 28(1988): 4 265-278; Tschopp et al., Bio/Technology 5(1987): 1305-1308; Cregg et al., Bio/Technology 5(1987): 479-485; Sreekrishna et al. Biochemistry 28(1989): 4117-4125; and Bolen et al., Yeast 10: 403-414 (1994).

General recombinant DNA methods can be found, for example, in Sambrook et al., Molecular Cloning: A Laboratory Manual (2nd ed., Cold Spring Harbor Laboratory Press, Plainview, N.Y., 1989).

All publications, patents and patent applications cited herein, whether supra or infra, are hereby incorporated by reference in their entirety.

### SUMMARY OF THE INVENTION

It is an object of the invention to provide a polynucleotide molecule comprising a first nucleotide sequence that encodes at least a fragment of a leader sequence and a second nucleotide sequence that encodes a polypeptide heterologous to the leader sequence,

wherein the leader sequence fragment is sufficient for secretion and comprises an amino acid sequence that comprises at least about 70% sequence identity to the leader sequence of Pichia acaciae killer toxin,

wherein the heterologous polypeptide is not naturally contiguous to the leader sequence, and

wherein upon expression of the polynucleotide molecule in a host cell suitable for expression thereof, the heterologous polypeptide is produced that is free of additional N-terminal amino acids.

The polynucleotide of the invention can be used to construct expression vectors and host cells capable of producing the polynucleotide or expressing the desired polypeptide.

Yet another object of the invention is to provide a method of producing a polypeptide encoded by a polynucleotide comprising

(a) transforming a host cell with the polynucleotide,

(b) allowing the expression thereof to produce the polypeptide and

(c) obtaining the polypeptide therefrom,

wherein the polynucleotide molecule comprises a first nucleotide sequence that encodes at least a fragment of a leader sequence and a second nucleotide sequence that encodes a polypeptide heterologous to the leader sequence,

wherein the leader sequence fragment is sufficient for secretion and comprises an amino acid sequence that comprises at least about 70% sequence identity to the leader sequence of Pichia acaciae killer toxin,

wherein the heterologous polypeptide is not naturally contiguous to the leader sequence, and

wherein upon expression of the polynucleotide molecule in a host cell suitable for expression thereof, the heterologous polypeptide is produced that is free of additional N-terminal amino acids.

A specific embodiment of the invention is where the heterologous polypeptide is human insulin-like growth factor 1 (IGF-1).

### BRIEF DESCRIPTION OF THE DRAWINGS

FIG. 1 is a plasmid map of pHIL-A1.

FIG. 2 shows the approximately 300 base-pair segment of the 3′ AOX1 (alcohol oxidase, OA) transcriptional termination region residing in pHIL-A1. The 3′ AOX1 contains a small stretch (22 amino acids long) of carboxy terminal alcohol oxidase coding sequences up to translational stop codon TAA (italicized and underlined). The 3′ end of AOX1 mRNA is in bold and is also underlined (A). The entire 341 base-pair sequence is set forth in SEQ ID NO:16.

FIG. 3 shows the approximately 750 base-pair segment of the 5′ AOX1 promoter residing in pHIL-A1. The alcohol oxidase coding sequence following the A of the ATG initiating methionine codon has been removed and a synthetic linker used to generate a unique EcoRI site, as described for pHIL-D1 (available from Invitrogen, San Diego, Calif.). Nucleotides added immediately following the “A” of the translation initiation codon to create this EcoRI site are italicized. The 5′ end of the alcohol oxidase mRNA has been denoted as a major species (*) or minor species (ˆ ) of mRNA transcripts. The entire 1018 base-pair sequence is set forth in SEQ ID NO:18.

FIG. 4 shows the nucleotide sequence of the 164 base-pair PARS1 Taq I fragment (SEQ ID NO: 17) of the Pichia pastoris PARS1 autonomous replication sequence residing in pHIL-A1.

### DETAILED DESCRIPTION

Definitions

“Heterologous” means not naturally contiguous. For example, a yeast leader and a human protein are heterologous because the two are not naturally contiguous.

A host cell suitable of “expression of a polynucleotide” is capable of effecting transcription and translation of the polynucleotide to produce the encoded heterologous polypeptide free of additional N-terminal amino acids.

General Methods and Detailed Description

Preferably, polynucleotides of the instant invention are produced by recombinant DNA techniques. The polynucleotide encoding at least a fragment of a leader sequence can be either synthesized or cloned.

The amino acid sequence of the leader sequence comprises at least 70% sequence identity to the leader sequence of the Pichia acaciae killer toxin, described in Bolen et al., Yeast 10: 403-414 (1994) and shown in SEQ ID NO:2. More preferably, the leader sequence comprises at least 80%; even more preferably, at least 90%; more preferably, at least 95% sequence identity to SEQ ID NO:2; most preferably, 100% sequence identity to SEQ ID NO:2.

A full-length leader sequence begins at the initiating methionine and ends at the last amino acid residue before the beginning of the encoded mature polypeptide. Amino acid residues can be removed from full-length leader to construct leader fragments. These fragments can be tested to determine if they are sufficient for secretion.

Empirical data can be used, for example, to determine if a fragment of a leader sequence is sufficient for secretion. Host cells with the polynucleotide of the instant invention exhibit increased expression levels as compared to a negative control. See below for assays to detect polypeptide expression.

A full-length leader sequence from a native gene, such a Pichia acaciae killer toxin, can be divided into a signal peptide region and a pro-region. Typically, a fragment sufficient for secretion comprises a signal peptide. Signal peptides are generally hydrophobic and exhibit a three dimensional helical structure. Also, a cleavage site can be incorporated in the fragment to facilitate removal of the leader fragment from the heterologous polypeptide. Examples are peptidase cleavage sites, which include KEX2 as an example. Preferably, the cleavage site comprises a dibasic dipeptide such as, lys-lys, arg-arg, more preferably lys-arg.

The leader sequence can be altered for convenience or to optimize expression. For example, the amino acid sequence of Pichia acaciae signal peptide can be mutated. The following are examples of conservative substitutions: Gly⇄Ala; Val⇄Ile⇄Leu; Asp⇄Glu; Lys⇄Arg; Asn⇄Gln; and Phe⇄Trp⇄Tyr. A subset of mutants, called muteins, is a group of polypeptides with the non-disulfide bond participating cysteines substituted with a neutral amino acid, generally, with serines.

The amino acid sequence of the Pichia acaciae killer toxin leader sequence of SEQ ID NO:2 can be aligned with the leader sequence of other yeast killer toxin genes to determine the positions of variable and conserved amino acid residues.

Full-length and fragments of Pichia acaciae killer toxin leader sequences as well as mutants thereof, can be fused with additional amino acid residues. For example, the consensus sequence of pro-regions from other leader sequences can be determined and incorporated into the leader sequence. Such pro-region sequences can be helpful to optimize expression in a particular host cell.

Polynucleotide sequence encoding the leader sequence can be based on the sequence found in genomic DNA or be made by using codons preferred by the host cell. In both cases, the polynucleotides can be synthesized using the methods described in Urdea et al., Proc. Natl. Acad. Sci. USA 80: 7461 (1983), for example. Alternatively, the polynucleotides from nucleic acid libraries using probes based on the nucleic acid sequence shown in SEQ ID NO:1. Techniques for producing and probing nucleic acid sequence libraries are described, for example, in Sambrook et al., “Molecular Cloning: A Laboratory Manual” (New York, Cold Spring Harbor Laboratory, 1989). Other recombinant techniques, such as site specific mutagenesis, PCR, enzymatic digestion and ligation, can also be used to clone or modify the sequences found from natural sources.

Similarly, the polynucleotides encoding the desired polypeptide can also be constructed using synthetic or recombinant means. Amino acid sequence of polypeptides to be expressed can also be found in publicly available databases.

Useful polypeptides to be expressed include, for example, hormones, growth factors, cytokines, haematopoietic factors, immunoglobulins, enzymes, repressors, cell differentiation factors, binding proteins, or transcription factors. Specific examples are: growth hormone, luteinizing hormone, thyroid stimulating hormone, oxytocin, insulin, vasopressin, renin, calcitonin, follicle stimulating hormone, prolactin, insulin-like growth factor (IGF-I, IGF-II), an IGF-binding protein, epidermal growth factor (EGF), platelet derived growth factor (PDGF), keratinocyte growth factor (KGF), fibroblast growth factor (FGF), nerve growth factor (NGF), TGF-beta, vascular endothelial cell growth factor (VEGF), erythropoietin (EPO), colony stimulating factor (CSF), interferon, endorphin, enkaphalin, dynorphin and an active fragment thereof.

The two polynucleotides, encoding at least a fragment of a leader sequence and the heterologous polypeptide, are linked together to produce the polynucleotide of the instant invention. Preferably, the polynucleotides are linked together in proper reading frame.

Polynucleotides encoding at least a fragment of a leader sequence and encoding polypeptides can be expressed by a variety of host cells. Although the leader sequence may be yeast derived and linked to a human protein, for example, host cells as diverse as yeast, insect, and mammalian host cells can express the polypeptide.

Typically, the polynucleotide of the instant invention, leader sequence and polypeptide, can be incorporated into an expression vector, which is in turn inserted into the desired host cell for expression.

At the minimum, an expression vector will contain a promoter which is operable in the host cell and operably linked to polynucleotide of the instant invention. Expression vectors may also include signal sequences, terminators, selectable markers, origins of replication, and sequences homologous to host cell sequences. These additional elements are optional but can be included to optimize expression.

A promoter is a DNA sequence upstream or 5′ to the polynucleotide of the instant invention to be expressed. The promoter will initiate and regulate expression of the coding sequence in the desired host cell. To initiate expression, promoter sequences bind RNA polymerase and initiate the downstream (3′) transcription of a coding sequence (e.g. structural gene) into mRNA. A promoter may also have DNA sequences that regulate the rate of expression by enhancing or specifically inducing or repressing transcription. These sequences can overlap the sequences that initiate expression. Most host cell systems include regulatory sequences within the promoter sequences. For example, when a repressor protein binds to the lac operon, an E. coli regulatory promoter sequence, transcription of the downstream gene is inhibited. Another example is the yeast alcohol dehydrogenase promoter, which has an upstream activator sequence (UAS) that modulates expression in the absence of glucose. Additionally, some viral enhancers not only amplify but also regulate expression in mammalian cells. These enhancers can be incorporated into mammalian promoter sequences, and the promoter will become active only in the presence of an inducer, such as a hormone or enzyme substrate (Sassone-Corsi and Borelli (1986) Trends Genet. 2:215; Maniatis et al. (1987) Science 236:1237).

Functional non-natural promoters may also be used, for example, synthetic promoters based on a consensus sequence of different promoters. Also, effective promoters can contain a regulatory region linked with a heterologous expression initiation region. Examples of hybrid promoters are the E. coli lac operator linked to the E. coli tac transcription activation region; the yeast alcohol dehydrogenase (ADH) regulatory sequence linked to the yeast glyceraldehyde-3-phosphate-dehydrogenase (GAPDH) transcription activation region (U.S. Pat. Nos. 4,876,197 and 4,880,734, incorporated herein by reference); and the cytomegalovirus (CMV) enhancer linked to the SV40 (simian virus) promoter.

Typically, terminators are regulatory sequences, such as polyadenylation and transcription termination sequences, located 3′ or downstream of the stop codon of the coding sequences. Usually, the terminator of native host cell proteins are operable when attached 3′ of the polynucleotide of the instant invention. Examples are the Saccharomyces cerevisiae alpha-factor terminator and the baculovirus terminator. Further, viral terminators are also operable in certain host cells; for instance, the SV40 terminator is functional in CHO cells.

For convenience, selectable markers, an origin of replication, and homologous host cell sequences may optionally be included in an expression vector. A selectable marker can be used to screen for host cells that potentially contain the expression vector. Such markers may render the host cell immune to drugs such as ampicillin, chloramphenicol, erythromycin, neomycin, and tetracycline. Also, markers may be biosynthetic genes, such as those in the histidine, tryptophan, and leucine biosynthetic pathways. Thus, when leucine is absent from the media, for example, only the cells with a biosynthetic gene in the leucine pathway will survive.

An origin of replication may be needed for the expression vector to replicate in the host cell. Certain origins of replication enable an expression vector to be reproduced at a high copy number in the presence of the appropriate proteins within the cell. Examples of origins are the 2m and autonomously replicating sequences, which are effective in yeast; and the viral T-antigen, effective in COS-7 cells.

Expression vectors may be integrated into the host cell genome or remain autonomous within the cell. Polynucleotide sequences homologous to sequences within the host cell genome may be needed to integrate the expression cassette. The homologous sequences do not always need to be linked to the expression vector to be effective. For example, expression vectors can integrate into the CHO genome via an unattached dihydrofolate reductase gene. In yeast, it is more advantageous if the homologous sequences flank the expression cassette. Particularly useful homologous yeast genome sequences are those disclosed in PCT WO90/01800, and the HIS4 gene sequences, described in Genbank, accession no. J01331.

The choice of promoter, terminator, and other optional elements of an expression vector will also depend on the host cell chosen. The invention is not dependent on the host cell selected. Convenience and the level of protein expression will dictate the optimal host cell. A variety of hosts for expression are known in the art and available from the American Type Culture Collection (ATCC). Bacterial hosts suitable for expression include, without limitation: Campylobacter, Bacillus, Escherichia, Lactobacillus, Pseudomonas, Staphylococcus, and Streptococcus. Yeast hosts from the following genera may be utilized: Candida, Hansenula, Kluyveromyces, Pichia, Saccharomyces, Schizosaccharomyces, and Yarrowia. Immortalized mammalian host cells include but are not limited to CHO cells, HeLa cells, baby hamster kidney (BHK) cells, monkey kidney cells (COS), human hepatocellular carcinoma cells (e.g., Hep G2), and other cell lines. A number of insect cell hosts are also available for expression of heterologous proteins: Aedes aegypti, Bombyx mori, Drosophila melanogaster, and Spodoptera frugiperda (PCT WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Transformation

After vector construction, the expression vector is inserted into the host cell. Many transformation techniques exist for inserting expression vectors into bacterial, yeast, insect, and mammalian cells. The transformation procedure to introduce the expression vector depends upon the host to be transformed.

Methods of introducing exogenous DNA into bacterial hosts are well-known in the art, and typically protocol includes either treating the bacteria with CaCl₂ or other agents, such as divalent cations and DMSO. DNA can also be introduced into bacterial cells by electroporation or viral infection. Transformation procedures usually vary with the bacterial species to be transformed. See e.g., (Masson et al. (1989) FEMS Microbiol. Lett. 60:273; Palva et al. (1982) Proc. Natl. Acad. Sci. USA 79:5582; EP Publ. Nos. 036 259 and 063 953; PCT WO 84/04541, Bacillus), (Miller et al. (1988) Proc. Natl. Acad. Sci. 85:856; Wang et al. (1990) J. Bacteriol. 172:949, Campylobacter), (Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids,” in Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia), (Chassy et al. (1987) FEMS Microbiol. Lett. 44:173 Lactobacillus); (Fiedler et al. (1988) Anal. Biochem 170:38, Pseudomonas); (Augustin et al. (1990) FEMS Microbiol. Lett. 66:203, Staphylococcus), (Barany et al. (1980) J. Bacteriol. 144:698; Harlander (1987) “Transformation of Streptococcus lactis by electroporation,” in Streptococcal Genetics (ed. J. Ferretti and R. Curtiss III); Perry et al. (1981) Infec. Inmun. 32:1295; Powell et al. (1988) Appl. Environ. Microbiol. 54:655; Somkuti et al. (1987) Proc. 4th Evr. Cong. Biotechnology 1:412, Streptococcus).

Transformation methods for yeast hosts are well-known in the art, and typically include either the transformation of spheroplasts or of intact yeast cells treated with alkali cations. Electroporation is another means for transforming yeast hosts. See for example, Methods in Enzymology, Volume 194, 1991, “Guide to Yeast Genetics and Molecular Biology.” Transformation procedures usually vary with the yeast species to be transformed. See e.g., (Kurtz et al. (1986) Mol. Cell. Biol. 6:142; Kunze et al. (1985) J. Basic Microbiol. 25:141; Candida); (Gleeson et al. (1986) J. Gen. Microbiol. 132:3459; Roggenkamp et al. (1986) Mol. Gen. Genet. 202:302; Hansenula); (Das et al. (1984) J. Bacteriol. 158:1165; De Louvencourt et al. (1983) J. Bacteriol. 154:1165; Van den Berg et al. (1990) Bio/Technology 8:135; Kluyveromyces); (Cregg et al. (1985) Mol. Cell. Biol. 5:3376; Kunze et al. (1985) J. Basic Microbiol. 25:141; U.S. Pat. Nos. 4,837,148 and 4,929,555; Pichia); (Hinnen et al. (1978) Proc. Natl. Acad. Sci. USA 75;1929; Ito et al. (1983) J. Bacteriol. 153:163 Saccharomyces); (Beach and Nurse (1981) Nature 300:706; Schizosaccharomyces); (Davidow et al. (1985) Curr. Genet. 10:39; Gaillardin et al. (1985) Curr. Genet. 10:49; Yarrowia).

Methods for introducing heterologous polynucleotides into mammalian cells are known in the art and include viral infection, dextran-mediated transfection, calcium phosphate precipitation, polybrene mediated transfection, protoplast fusion, electroporation, encapsulation of the polynucleotide(s) in liposomes, and direct microinjection of the DNA into nuclei.

The method for construction of an expression vector for transformation of insect cells for expression of recombinant protein herein is slightly different than that generally applicable to the construction of a bacterial expression vector, a yeast expression vector, or a mammalian expression vector. In an embodiment of the present invention, a baculovirus vector is constructed in accordance with techniques that are known in the art, for example, as described in Kitts et al., BioTechniques 14: 810-817 (1993), Smith et al., Mol. Cell. Biol. 3: 2156 (1983), and Luckow and Summer, Virol. 17: 31 (1989). In one embodiment of the present invention, a baculovirus expression vector is constructed substantially in accordance to Summers and Smith, Texas Agricultural Experiment Station Bulletin No. 1555 (1987). Moreover, materials and methods for baculovirus/insect cell expression systems are commercially available in kit form, for example, the MaxBac® kit from Invitrogen (San Diego, Calif.).

Also, methods for introducing heterologous DNA into an insect host cell are known in the art. For example, an insect cell can be infected with a virus containing a coding sequence. When the virus is replicating in the infected cell, the polypeptide will be expressed if operably linked to a suitable promoter. A variety of suitable insect cells and viruses are known and include following without limitation.

Insect cells from any order of the Class Insecta can be grown in the media of this invention. The orders Diptera and Lepidoptera are preferred. Example of insect species are listed in Weiss et al., “Cell Culture Methods for Large-Scale Propagation of Baculoviruses,” in Granados et al. (eds.), The Biology of Baculoviruses: Vol. II Practical Application for Insect Control, pp. 63-87 at p. 64 (1987). Insect cell lines derived from the following insects are exemplary: Carpocapsa pomeonella (preferably, cell line CP-128); Trichoplusia ni (preferably, cell line TN-368); Autograph californica; Spodoptera frugiperda (preferably, cell line Sf9); Lymantria dispar; Mamestra brassicae; Aedes albopictus; Orgyia pseudotsugata; Neodiprio sertifer; Aedes aegypti; Antheraea eucalypti; Gnorimoschema operceullela; Galleria mellonella; Spodoptera littolaris; Blatella germanic; Drosophila melanogaster; Heliothis zea; Spodoptera exigua; Rachiplusia ou; Plodia interpunctella; Amsaeta moorei; Agrotis c-nigrum, Adoxophyes orana; Agrotis segetum; Bombyx mori; Hyponomeuta malinellu;, Colias eurytheme; Anticarsia germmetalia; Apanteles melanoscelu; Arctia caja; and Porthetria dispar. Preferred insect cell lines are from Spodoptera frugiperda, and especially preferred is cell line Sf9. The Sf9 cell line used in the examples herein was obtained from Max D. Summers (Texas A & M University, College Station, Tex., 77843, U.S.A.) Other S. frugiperda cell lines, such as IPL-Sf-21AE III, are described in Vaughn et al., In Vitro 13: 213-217 (1977).

The insect cell lines of this invention are suitable for the reproduction of numerous insect-pathogenic viruses such as parvoviruses, pox viruses, baculoviruses and rhabdcoviruses, of which nucleopolyhedrosis viruses (NPV) and granulosis viruses (GV) from the group of baculoviruses are preferred. Further preferred are NPV viruses such as those from Autographa spp., Spodoptera spp., Trichoplusia spp., Rachiplusia spp., Gallerai spp., and Lymantria spp. More preferred are baculovirus strain Autographa californica NPV (AcNPV), Rachiplusia ou NPV, Galleria mellonella NPV, and any plaque purified strains of AcNPV, such as E2, R9, S1, M3, characterized and described by Smith et al., J Virol 30: 828-838 (1979); Smith et al., J Virol 33: 311-319 (1980); and Smith et al., Virol 89: 517-527 (1978).

Typically, insect cells Spodoptera frugiperda type 9 (SF9) are infected with baculovirus strain Autographa californica NPV (AcNPV) containing a coding sequence. Such a baculovirus is produced by homologous recombination between a transfer vector containing the coding sequence and baculovirus sequences and a genomic baculovirus DNA. Preferably, the genomic baculovirus DNA is linearized and contains a disfunctional essential gene. The transfer vector, preferably, contains the nucleotide sequences needed to restore the disfunctional gene and a baculovirus polyhedrin promoter and terminator operably linked to the polynucleotides of the instant invention. (See Kitts et al., BioTechniques 14(5): 810-817 (1993).

The transfer vector and linearized baculovirus genome are transfected into SF9 insect cells, and the resulting viruses probably containing the desired coding sequence. Without a functional essential gene the baculovirus genome cannot produce a viable virus. Thus, the viable viruses from the transfection most likely contain the coding sequence and the needed essential gene sequences from the transfer vector. Further, lack of occlusion bodies in the infected cells are another verification that the coding sequence was incorporated into the baculovirus genome.

The essential gene and the polyhedrin gene flank each other in the baculovirus genome. The coding sequence in the transfer vector is flanked at its 5′ with the essential gene sequences and the polyhedrin promoter and at its 3′ with the polyhedrin terminator. Thus, when the desired recombination event occurs the coding sequence displaces the baculovirus polyhedrin gene. Such baculoviruses without a polyhedrin gene will not produce occlusion bodies in the infected cells. Of course, another means for determining if coding sequence was incorporated into the baculovirus genome is to sequence the recombinant baculovirus genomic DNA. Alternatively, expression of the desired polypeptide by cells infected with the recombinant baculovirus is another verification means.

Once transformed the host cells can be used to produce either polynucleotides of the instant invention or express the desired polypeptide.

Simple gel electrophoresis techniques can be used to detect expression of the desired polypeptide. For example, media from a host cell without an expression vector can be compared to media from host cell with the desired vector. Polyacrylamide gel electrophoresis (“PAGE”) can be used to determine if any proteins were expressed. Antibodies to the desired proteins can be used in Western blots to determine with greater sensitivity if protein was expressed.

### EXPERIMENTAL

The examples presented below are provided as a further guide to the practitioner of ordinary skill in the art, and are not to be construed as limiting the invention in any way.

### EXAMPLE 1

Construction of Pichia pastoris autonomously replicating vector containing P. pastoris HIS4 gene (SEQ ID NO: 19) as a selectable marker and an expression cassette (SEQ ID NO:13) containing a P. acaciae killer toxin leader and IGF-1 gene.

A. CLONING

I. Killer Toxin Leader Fragment

Construction of fragment by annealing of synthetic oligomers.

Synthesis of oligomers with a phosphate group attached or kinase.

The sequences of the oligomers KAC 34, KAC 37, KAC 39, KAC 59, KAC 60, and KAC 61 are set forth in SEQ ID NOs:3, 4, 5, 6, 7, and 8, respectively. Ligation of fragment and base vector for sequencing and ease of handling.

Fragment: as described above

Base vector: pLITMUS28 available from New England Biolabs (Beverly, Mass.)

II. IGF-1 Fragment

Isolation: from a yeast strain with an integrated vector. Sequence of gene attached.

III. Overlapping PCR

Construction of a single fragment containing the leader sequence and IGF-1 gene.

PCR #1:

Reaction Mix:

4 μL of IGF-1 gene fragment for a total of 10 ng

10 μL of Pfu DNA Polymerase buffer available from Stratagene (La Jolla, Calif.)

4 μL of a 2 mM dNTP

20 μL of oligomer KAC58 (SEQ ID NO:12) for a total of 20 picomoles

20 μL of oligomer KAC57 (SEQ ID NO:11) for a total of 20 picomoles

1 μL of 2.5 units/μL Pfu DNA Polymerase available from Stratagene (La Jolla, Calif., USA)

41 μL of water

Temperature cycle:

5 cycles: 97° C. for 1 minute, 43° C. for 1 minute, and 72° C. for 1 minute

24 cycles: 97° C. for 1 minute and 72° C. for 1 minute

PCR#2

Reaction Mix

1 μL of Killer toxin fragment in pLITMUS28 for a total of 10 ng

10 μL of 10× PCR buffer

2 μL of 2 mM dNTP

10 μL of oligomer KAC74 (SEQ ID NO:9) for a total of 10 picomoles

10 μL of oligomer KAC75 (SEQ ID NO:10) for a total of 10 picomoles

0.5 μL of 5 units/μL taq DNA Polymerase available from Boehringer Mannheim catalog number 1 146 173 (Indianapolis, Ind.)

66.5 μL of H₂O

10× PCR buffer

0.25M Tris-HCl, pH 8.3

0.015M MgCl₂ in 0.0015M EDTA

0.25M KCl

0.5% Tween 20

Temperature cycle:

5 cycles: 97° C. for 1 minute, 63° C. for 1 minute, and 72° C. for 1 minute

19 cycles: 97° C. for 1 minute and 72° C. for 1 minute

PCR #3

Reaction Mix:

5 μL of result PCR#2

5 μL of 1:100 dilution of result of PCR#1

10 μL of 10× Pfu DNA Polymerase buffer available from Stratagene (La Jolla, Calif., USA)

4 μL of 2 mM dNTP

1 μL of 2.5 units/μL of Pfu DNA Polymerase available from Stratagene(La Jolla, Calif.)

2 μL of oligomer KAC74 (SEQ ID NO:9) for a total of 2 picomoles

2 μL of oligomer KAC57 (SEQ ID NO:11) for a total of 2 picomoles

71 μL of water.

Temperature Cycle:

5 cycles: 97° C. for 1 minute, 58° C. for 1 minute, and 72° C. for 1 minute

24 cycles: 97° C. for 1 minute and 72° C. for 1 minute.

PCR#4

Reaction Mix:

1 μL of results of PCR#3

10 μL of KAC74 (SEQ ID NO:9) for a total of 10 picomoles

30 μL of KAC57 (SEQ ID NO: 11) for a total of 10 picomoles

10 μL of 10× PCR buffer (same as used in PCR#2)

2 μL of 2 mM dNTP

5 μL of 0.5 units/μL of taq DNA Polymerase available from Boehringer Mannheim catalog number 1 146 173 (Indianapolis, Ind., USA)

42 μL of water.

Temperature Cycle:

24 cycles: 97° C. for 1 minute and 72° C. for 1 minute

Ligation of PCR#4 Fragment to a Shuttle Vector for Sequencing

Fragment: 1 μL of result of PCR#4

Base vector: 2 μL of pCRII from Invitrogen (San Diego, Calif.)

Ligase: 1 μL from Invitrogen (San Diego, Calif.) kit #45-0046

10× Ligase buffer: 1 μL from Invitrogen (San Diego, Calif.) kit #45-0046

Water: 5 μL

Ligation into Expression

Base Vector: 2 μL of pHIL-A1, linear with EcoRI ends and dephosphorylated

Fragment: 2 μL of EcoRI from pCRII with expression cassette containing a killer toxin leader fragment with IGF-1 gene

Ligase: 1 μL of T4 DNA ligase available from Boerhinger Mannheim

10× Ligase buffer: 1 μL available from Boerhinger Mannheim

Water: 4 μL

Verification that expression cassette in correct orientation by restriction endonuclease mapping. The nucleotide sequence for this expression cassette is set forth in SEQ ID NO:13, and the amino acid sequence for the encoded killer toxin leader fragment with IGF-I gene fragment is set forth in SEQ ID NO:14. SEQ ID NO:15 sets forth the C-terminal peptide fragment encoded by nucleotides 376-390 of the expression cassette set forth in SEQ ID NO:13.

Description of pHIL-A1

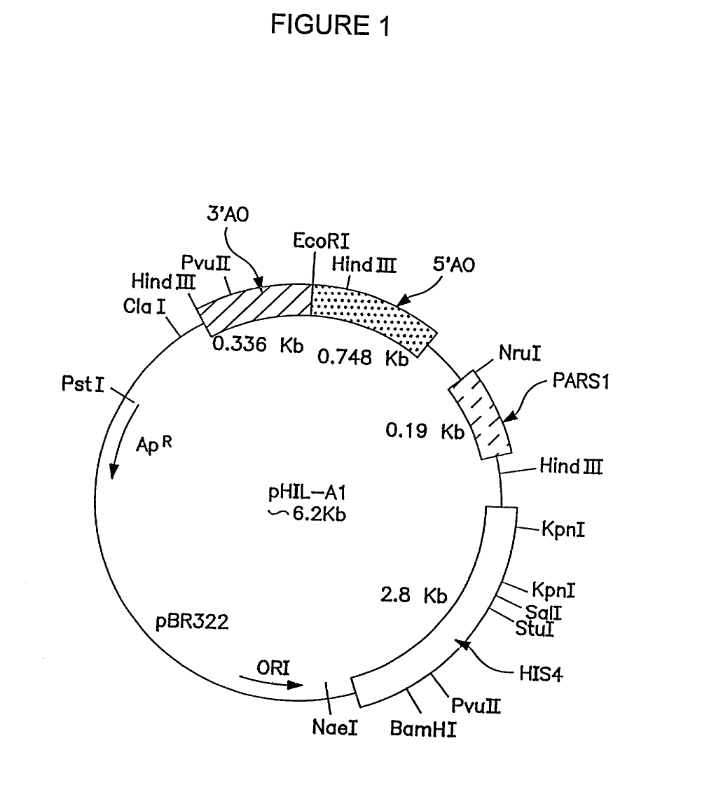

Plasmid pHIL-A1 is an E. coil- P. pastoris shuttle vector, with sequences for selection and autonomous replication in each host (see FIG. 1). One component of the plasmid is a modified portion of plasmid pBR322 containing the ampicillin resistance gene and the origin of replication (ori). The regions between nucleotides 1,100 and 2,485 of pBR322 and between NaeI sites 404 and 932 were deleted to eliminate “poison sequences” and the Sal I site, respectively.

The DNA elements comprising the rest of the plasmid are derived from the genome of P. pastoris, except for short regions of pBR322 used to the link the yeast elements. The yeast elements are as follows, proceeding clockwise:

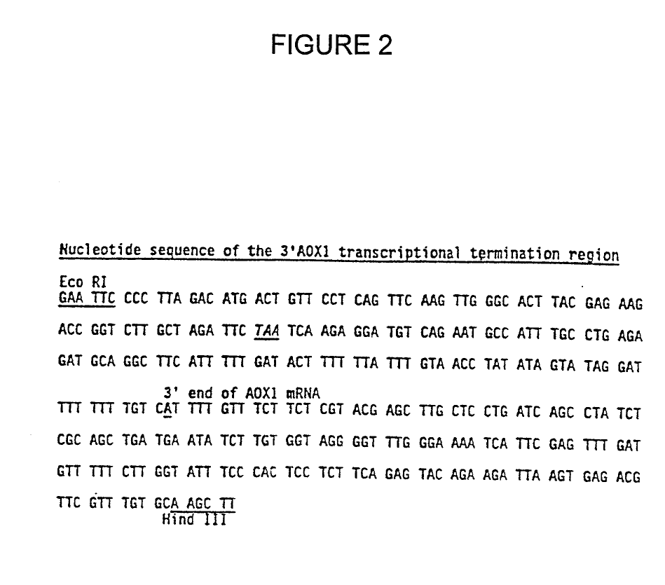

1. 3′ AOX1, alcohol oxidase, approximately 300 bp segment of the AO terminating sequence. See FIG. 2 and SEQ ID NO:16.

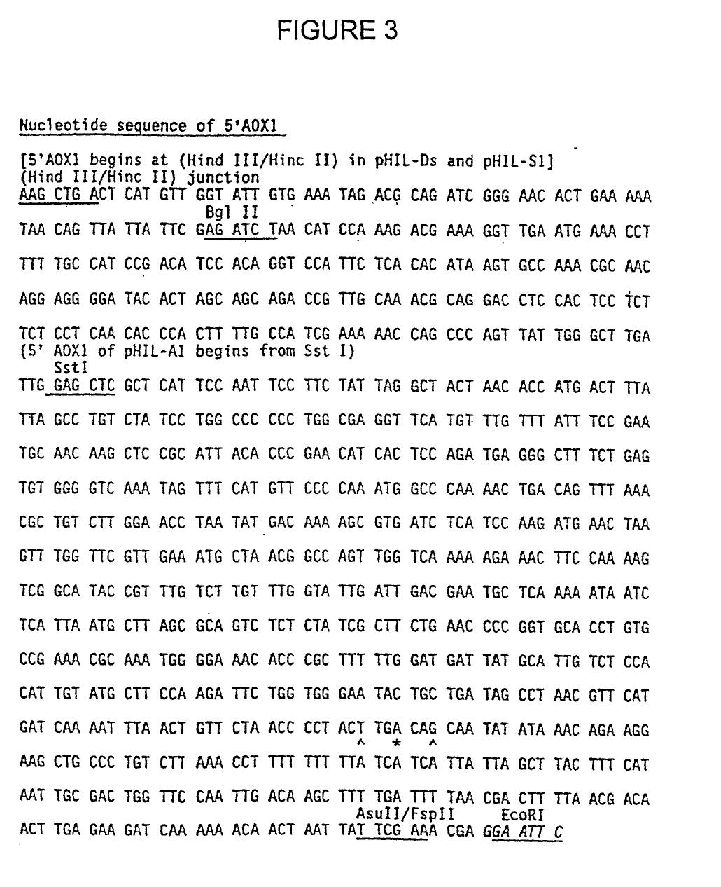

2. 5′ AOX1, approximately 750 bp segment of the alcohol oxidase promoter. The alcohol oxidase coding sequences following the A of the ATG initiating methionine codon have been removed, and a synthetic linker used to generate a unique EcoRl site, as described for pHIL-D1 (available from Invitrogen, San Diego, Calif.). See FIG. 3 and SEQ ID NO:18.

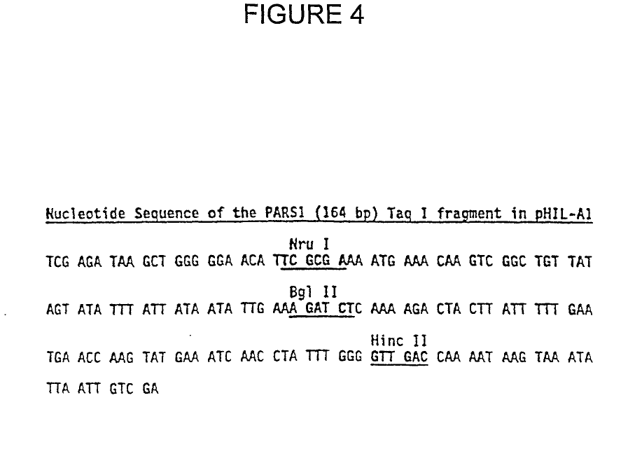

3. PARS1, approximately 190 bp segment of P. pastoris autonomous replication sequence. See FIG. 4 and SEQ ID NO:17.

4. HIS4, approximately 2.8 kb segment of P. pastoris histidinol dehydrogenase gene to complement the defective his4 gene in P. pastoris, strain GS115. See SEQ ID NO:19.

B. TRANSFORMATION

I. YEAST STRAIN

P. pastoris, GS115 available from Invitrogen (San Diego, Calif.), also available from the USDA, Northern Regional Research Center in Peoria, Ill., under the accession number NRRL Y-15851

or

P. pastoris SMD1163

II. ELECTROPORATION

Cells: Cells from preculture at approximately 16 OD_{600.} 1:20 dilution into 10% glycerol with water. 50 μL of cells in 10% glycerol with water for electroporation.

Equipment:

BioLab Pulse Controller and BioLab Gene Pulser

Pulse:

2.0 Kilovolts

25 μFD

200 ohms

Time Constant:

5 Milliseconds

Selection:

Cells on minimal medium in minus histidine with glucose

C. EXPRESSION

I. Precultures

Media:

Minimal his minus media plus glucose

Inoculum:

One transformed colony

Temperature:

30° C.

Time: until culture is saturated

II. Expression Cultures

Media: 25 mL of MGY

MGY=

13. g/L of Yeast Nitrogen Base without amino acids, available from Difco (Mich., Detroit, USA)

400 μg/L biotin

1% (v/v) glycerol

0.1% leucine

0.1% lysine

0.1% tryptophan

0.1% adenine

0.1 % uracil

Inoculum:

250 μL of the preculture

Temperature

30° C.

Aeration:

275 rpm

Time:

Approximately 48 hours or 5-10 OD₆₀₀

Harvest:

4000 rpm for 10 minutes

Wash, Resuspension, and Dilution of cells:

Use MM media for all.

MM=

13. g/L of Yeast Nitrogen Base without amino acids, available from Difco (Mich., Detroit, USA)

400 μg/L biotin

0.5% (v/v) methanol

0.1% leucine

0.1% lysine

0.1% tryptophan

0.1% adenine

0.1% uracil

Resuspension: with approximately 5 mL

Dilution: to approximately 3 OD₆₀₀.

Temperature:

30° C.

Aeration:

275 rpm

Time:

Approximately 96 hours

### EXAMPLE 2

Construction of Pichia pastoris integrating vector containing P. pastoris HIS4 gene (SEQ ID NO:19) as a selectable marker and multiple copies of an expression cassette (SEQ ID NO:13) containing the P. acaciae leader and IGF1 gene.

STAGE 1 CLONING:

Starting vector:

pA0815 as described by Brierley et al., U.S. Pat. No. 5,324,639 and available from Invitrogen (San Diego, Calif.). The vector contains a unique EcoRI restriction site flanked by the P. pastoris alcohol oxidase 1 (“AO1”) gene promoter and terminator.

Insert Fragment:

Described above in Example 1 comprising EcoRI restriction ends.

Resulting vector 1:

One AO1 gene promoter

One P. acaciae killer toxin leader

One IGF-1 gene

One AO1 gene terminator.

STAGE 2 CLONING:

Fragment:

BglII-BamHI fragment from Resulting vector 1.

Base vector:

The entire resulting vector 1, linear with BamHI ends

Resulting vector 2:

pALIGF1-2 with two expression cassettes each with

One AO1 gene promoter

One P. acaciae killer toxin leader

One IGF-1 gene

One AO1 gene terminator.

STAGE 3:

Fragment:

BglII-BamHI fragment from Resulting vector 2, pALIGF1-2.

Base Vector:

The entire pALIGF1-2, linear with BamHI ends

Resulting Vector:

pALIGF1-3 with four expression cassettes with

One AO1 gene promoter

One P. acaciae killer toxin leader

One IGF-1 gene

One AO1 gene terminator.

STAGE 4:

Fragment:

BglII-BamHI fragment from Resulting vector 2, pALIGF1-2.

Base Vector:

The entire pALIGF1-3, linear with BamHI ends

Resulting Vector:

pALIGF1-4 with six expression cassettes with

One AO1 gene promoter

One P. acaciae killer toxin leader

One IGF-1 gene

One AO1 gene terminator.

TRANSFORMATION:

Yeast:

P. pastoris, GS115, available from Invitrogen (San Diego, Calif.) or P. pastoris, SMD 1163.

Electroporation: Same as Example 1.

Expression: Same as Example 1.

### EXAMPLE 3

### Construction of Three Vectors, pKK, pKG, and pKGK

These vectors comprise the IGF-1 coding sequence. Further, the vectors comprise killer toxin leader sequences as described below:

(The asterisks indicate the amino acid positions that are different from the native killer toxin sequence.)

pKG=killer toxin leader with glycosylation site, sequence below:

Met-Leu-Ile-Ile-Val-Leu-leu-Phe-Leu-Ala-Thr-Leu-Ala-Asn-Ser-Leu-Asp-Cys-Ser-Gly -Asp-Val-Phe-Phe-Gly-Tyr-Thr-Arg-Gly-Asp-Lys-Thr-Asp-Val-His-Lys-Ser-Gln-Asn* -Leu-Thr-Ala-Val-Lys-Asn-Ile-Lys-Arg- (SEQ ID NO:21)

pKK=killer toxin with KEX2 site, sequence below:

Met-Leu-Ile-Ile-Val-Leu-leu-Phe-Leu-Ala-Thr-Leu-Ala-Asn-Ser-Leu-Asp-Cys-Ser-Gly -Asp-Val-Phe-Phe-Gly-Tyr-Thr-Arg-Gly-Asp-Lys-Thr-Asp-Val-His-Lys-Ser-Gln-Ala -Leu-Thr-Ala-Val-Pro*-Met*-Tyr*-Lys-Arg (SEQ ID NO:23)

pKGK=killer toxin with glycosylation site and KEX2 site, sequence below:

Met-Leu-Ile-Ile-Val-Leu-leu-Phe-Leu-Ala-Thr-Leu-Ala-Asn-Ser-Leu-Asp-Cys-Ser-Gly -Asp-Val-Phe-Phe-Gly-Tyr-Thr-Arg-Gly-Asp-Lys-Thr-Asp -Val-His-Lys-Ser-Gln-Asn* -Leu-Thr-Ala-Val-Pro*-Met*-Tyr*-Lys-Arg ( SEQ ID NO:22)

A. ANNEALING OLIGOMERS

Construction of killer toxin fragments by annealing of synthetic oligomers. The DNA oligomers comprise a 5′ phosphate group. The sequences of the oligomers, KAC117, KAC118, KAC119, KAC120, KAC121, KAC122, KAC123, KAC124, KAC129, KAC130, KAC131, KAC132, KAC125, KAC126, KAC127, KAC128, and KAC133 are set forth in SEQ ID NOs:24-40, respectively.

Oligomers were diluted to a concentration of 100 picomoles in final volume of 500 μl with 5 μl polyA (1 mg/mL) and 50 μl of 10× ligase buffer. (Ligase buffer purchased from New England Biolabs, Beverly, Mass.).

| | PKK | | pKG. | pKGK | pmoles/μl |
|---|---|---|---|---|---|
| KAC117 | 4.8 | μL | 4.8 | 4.8 | 20.7 |
| KAC122 | 2.9 | | 2.9 | 2.9 | 34.3 |
| KAC118 | 4.5 | | 4.5 | 4.5 | 22.1 |
| KAC123 | 5 | | 5 | 5. | 20.0 |
| KAC119 | 3.8 | | 3.8 | 3.8 | 26.3 |
| KAC124 | 4.6 | | 4.6 | 4.6 | 21.5 |
| KAC120 | 3.5 | | 3.5 | 3.5 | 28.4 |
| KAC125 | 4 | | 4 | 4 | 24.9 |
| KAC121 | 5.4 | | 5.4 | 5.4 | 18.4 |
| KAC126 | 2.1 | | 2.1 | 2.1 | 46.6 |
| KAC109 | 1 | | 1 | 1 | |
| KAC127 | 9 | | 9 | 9 | 11.1 |
| KAC128 | | | 3.6 | | 27.8 |
| KAC129 | | | 3.6 | | 27.6 |
| KAC130 | 3.5 | | | | 28.8 |
| KAC131 | 4.1 | | | | 24.4 |
| KAC132 | | | | 2.2 | 44.3 |
| KAC133 | | | | 4.4 | 22.9 |

Oligomer mixtures were incubated for two minutes in boiling water. The mixture was cooled to room temperature (˜3 hours) with a little ice in bath, which was removed from the heat source.

LIGATION INTO YEAST VECTOR:

The following is the ligation mixture used to construct the leader/coding sequences:

2 μL of 10× ligation solution with ATP

2 μL of a fragment from pHIL-A1 vector digested with EcoRI and phosphotased for a total of 30 ng (plasmid described above)

1 μL of T4 DNA ligase for a total of 1 one unit

q.s. to final volume of 20 μL with water.

Either 1 μL or 5 μL of the above three oligomer mixtures were used for the ligation.

Incubated overnight at 4° C.

TRANSFORMATION INTO YEAST HOST

The vectors were transformed into Pichia pastoris yeast host, SMD1163, available from Invitrogen (San Diego, Calif.).

Before transformation, 3 mL of YEPD was inoculated with P. pastoris SMD1163. This culture was incubated overnight. Ten microliters of this overnight culture was used to inoculate 100 mL of YEPD.

These cells were grown to an OD₆₅₀ of 0.78. Then, the cells were centrifuged for 5 minutes at 3.5K. Cell pellets were resuspended in 100 mL sterile water. The cells were centrifuged for 5 minutes at 3.5K. The cell pellets were resuspended in 8 mL of 0.1M lithium acetate.

The cells were incubated in the lithium acetate for 30 minutes at 30° C. while shaking. Next, the cells were centrifuged again for 5 minutes in a table-top centrifuge and the cell pellets were resuspended in 8 mL of 0.1M lithium acetate.

Ten microliters of either pKK, pKG, or pKGK at 1 μg/mL was added to 100 μL of the cells in 0.1M lithium acetate. The cells and DNA were incubated for 30 minutes at 30° C.

Next, 0.6 mL of 40% PEG 3550, was added to the cells and DNA. The mixture was vortexed, and the mixture was incubated for 60 minutes at 30° C.

Then, the cells were centrifuged for 30 seconds and the cell pellets were resuspended in 60 μL of water. The mixture was plated on histidine minus, yeast minimal media.

Deposit Information

The following materials were deposited with the American Type Culture Collection:

| Name | Deposit Date | Accession No. |
|---|---|---|
| Escherichia coli XL1 | 26 Sept 1995 | 69903 |
| Blue pHIL-A1 paKT | | |

The above materials have been deposited with the American Type Culture Collection, Rockville, Md., under the accession numbers indicated. This deposit will be maintained under the terms of the Budapest Treaty on the International Recognition of the Deposit of Microorganisms for purposes of Patent Procedure. The deposits will be maintained for a period of 30 years following issuance of this patent, or for the enforceable life of the patent, whichever is greater. Upon issuance of the patent, the deposits will be available to the public from the ATCC without restriction.

These deposits are provided merely as convenience to those of skill in the art, and are not an admission that a deposit is required under 35 U.S.C. §112. The sequence of the polynucleotides contained within the deposited materials, as well as the amino acid sequence of the polypeptides encoded thereby, are incorporated herein by reference and are controlling in the event of any conflict with the written description of sequences herein. A license may be required to make, use, or sell the deposited materials, and no such license is granted hereby.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed:

1. A polynucleotide molecule comprising a first nucleotide sequence that encodes at least a fragment of a leader sequence and a second nucleotide sequence that encodes a polypeptide heterologous to the leader sequence,
wherein the leader sequence fragment is sufficient for secretion and comprises an amino acid sequence that comprises 100% sequence identity to the amino acid sequence set forth as amino acid residues 1-48 of SEQ ID NO:2,
wherein the heterologous polypeptide is not naturally contiguous to the leader sequence, and
wherein upon expression of the polynucleotide molecule in a host cell suitable for expression thereof, the heterologous polypeptide is produced that is free of additional N-terminal amino acids.

2. The polynucleotide molecule of claim 1, wherein the host cell is an eukaryotic cell.

3. The polynucleotide molecule of claim 2, wherein the eukaryotic cell is a yeast cell.

4. The polynucleotide molecule of claim 3, wherein the yeast cell belongs to a genus that is selected from the genera consisting of Pichia, Saccharomyces, Kluyveromyces, and Hansenula.

5. The polynucleotide of claim 4, wherein the yeast cell is selected from the group consisting of Pichia pastoris, Saccharomyces cerevisiae, Kluyveromyces lactis, and Hansenula polymorpha.

6. The polynucleotide molecule of claim 1, wherein the host cell is a protease A deficient cell.

7. The polynucleotide molecule of claim 1, wherein the host cell is a protease B deficient cell.

8. The polynucleotide molecule of claim 1, wherein the host cell is a protease A and protease B deficient cell.

9. The polynucleotide molecule of claim 1, wherein the leader sequence comprises a signal peptide sequence and a peptidase cleavage site that comprises dibasic amino acid residues.

10. The polynucleotide of claim 1, wherein the polynucleotide is DNA.

11. The polynucleotide of claim 1, wherein the polynucleotide is RNA.

12. An expression vector comprising the polynucleotide of claim 1, wherein the vector replicates independently or integrates into a host genome.

13. A host cell comprising the polynucleotide of claim 1, wherein the host cell effects transcription and translation of the polynucleotide to produce the heterologous polypeptide.

14. A host cell comprising the vector of claim 12 wherein the host cell effects transcription and translation of the polynucleotide to produce the heterologous polypeptide.

15. A method of producing a polypeptide comprising culturing the host cell of claim 13 and obtaining the polypeptide molecule therefrom.

16. A method of producing the polynucleotide molecule of claim 1, comprising linking together in proper reading frame the first nucleotide sequence and the second nucleotide sequence.

17. A method of producing the vector of claim 12, wherein the vector replicates independently, comprising linking together in proper reading frame a replicon and a polynucleotide molecule,
wherein the polynucleotide molecule comprises a first nucleotide sequence that encodes at least a fragment of a leader sequence and a second nucleotide sequence that encodes a polypeptide heterologous to the leader sequence,
wherein the leader sequence fragment is sufficient for secretion and comprises an amino acid sequence that comprises at least about 100% sequence identity to the amino acid sequence set forth as amino acid residues 1-48 of SEQ ID NO:2,
wherein the heterologous polypeptide is not naturally contiguous to the leader sequence and wherein upon expression of the polynucleotide molecule in a host cell suitable for expression thereof, the heterologous polypeptide is produced that is free of additional N-terminal amino acids.

18. The host cell of claim 13, wherein the cell is selected from the group consisting of a prokaryotic cell and a eukaryotic cell.

19. The host cell of claim 18, wherein the host cell is a eukaryotic cell and the eukaryotic cell is selected from the group consisting of a yeast cell, an avian cell, an insect cell, and a mammalian cell.

20. The host cell of claim 19, wherein the cell is a yeast cell, and the yeast cell is selected from the genera consisting of Pichia, Saccharomyces, and Kluyveromyces.

21. The host cell of claim 20, wherein the yeast cell is selected from the group consisting of Pichia pastoris, Saccharomyces cerevisiae, and Kluyveromyces lactis.

22. The polynucleotide of claim 1, wherein the heterologous polypeptide is a mammalian polypeptide.

23. The polynucleotide of claim 22, wherein the mammalian polypeptide is a human polypeptide.

24. The polynucleotide of claim 1, wherein the polypeptide is one selected from the group consisting of a hormone, a growth factor, a cytokine, a haematopoietic factor, an immunoglobulin, an enzyme, a repressor, a cell differentiation factor, a binding protein, and a transcription factor.

25. The polynucleotide of claim 1, wherein the polypeptide is one selected from the group consisting of growth hormone, luteinizing hormone, thyroid stimulating hormone, oxytocin, insulin, vasopressin, renin, calcitonin, follicle stimulating hormone, prolactin, insulin-like growth factor (IGF-I, IGF-II), an IGF-binding protein, epidermal growth factor (EGF), platelet derived growth factor (PDGF), keratinocyte growth factor (KGF), fibroblast growth factor (FGF), nerve growth factor (NGF), TGF-beta, vascular endothelial cell growth factor (VEGF), erythropoietin (EPO), colony stimulating factor (CSF), interferon, endorphin, enkaphalin, dynorphin, and active fragments thereof.

26. A method of producing a polypeptide encoded by a polynucleotide comprising
(a) transforming a host cell with the polynucleotide,
(b) allowing the expression thereof to produce the polypeptide and
(c) obtaining the polypeptide therefrom,
wherein the polynucleotide molecule comprises a first nucleotide sequence that encodes at least a fragment of a leader sequence and a second nucleotide sequence that encodes a polypeptide heterologous to the leader sequence,
wherein the leader sequence fragment is sufficient for secretion and comprises an amino acid sequence that comprises at least about 100% sequence identity to the amino acid sequence set forth as amino acid residues 1-48 of SEQ ID NO:2,
wherein the heterologous polypeptide is not naturally contiguous to the leader sequence, and
wherein upon expression of the polynucleotide molecule in a host cell suitable for expression thereof, the heterologous polypeptide is produced that is free of additional N-terminal amino acids.
